(19) United States
(12) Patent Application Publication
(10) Pub. No.: US 2008/0255351 A1
(43) Pub. Date: Oct. 16, 2008

(54) Method for Preparing Aminodiglycol (Adg) and Morpholine

(75) Inventors: Bram Willem Hoffer, Heidelberg (DE); Holger Evers, Munchen (DE); Petr Kubanek, Mannheim (DE); Till Gerlach, Ludwigshafen (DE); Johann-Peter Melder, Bohl-Iggelheim (DE); Frank Funke, Shanghai (CN); Matthias Frauenkron, Kalmhout (NL); Helmut Schmidtke, Bensheim (DE)

Correspondence Address: CONNOLLY BOVE LODGE & HUTZ, LLP, P O BOX 2207, WILMINGTON, DE, 19899, US

(73) Assignee: BASF AKTIENGESELLSCHAFT, Ludwigshafen (DE)

(21) Appl. No.: 12/088,718

(22) PCT Filed: Sep. 25, 2006
(86) PCT No.: PCT/EP2006/066665

§ 371 Date: Mar. 31, 2008

(30) Foreign Application Priority Data
Sep. 30, 2005 (DE) 10 2005 047 458.6
Feb. 6, 2006 (EP) 06101339.7

(51) Int. Cl. C07D 265/30

(52) U.S. Cl. 544/106

(57) ABSTRACT

Processes comprising: providing a starting material comprising diethylene glycol; and reacting the starting material with ammonia in the presence of a heterogeneous transition metal catalyst to form a reaction product comprising aminodiglycol and morpholine; wherein the catalyst comprises a catalytically active composition, which prior to treatment with hydrogen, comprises a mixture of oxygen-containing compounds of copper, nickel, cobalt and at least one of aluminum and zirconium; and wherein the catalyst is present as one or more shaped catalyst particles selected from spheres, extrudates, pellets and other geometries, wherein the sphere or extradate has a diameter of <3 mm, the pellet has a height of <3 mm, and the other geometries have an equivalent diameter L=1/a′ of <0.70 mm, where a′ is the external surface area per unit volume (mmₛ²/mmₚ³), as defined by

$${a}^{′} = \frac{{A}_{p}}{{V}_{p}}$$

where Aₚ is the external surface area of the catalyst particle (mmₛ²) and Vₚ is the volume of the catalyst particle (mmₚ³).

The present invention relates to a process for preparing aminodiglycol (ADG) and morpholine by reacting diethylene glycol (DEG) of the formula

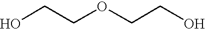

with ammonia in the presence of a heterogeneous transition metal catalyst.

Aminodiglycol (ADG) and morpholine are used, inter alia, as solvents, stabilizers, for the synthesis of chelating agents, synthetic resins, drugs, inhibitors and surface-active substances.

Numerous methods have been described in the literature for preparing aminodiglycol (ADG) and morpholine.

EP-A-36 331 and U.S. Pat. No. 4,647,663 describe a process for preparing morpholine and morpholine derivatives by reacting a dialkylene glycol with ammonia in the presence of H₂ and a hydrogenation catalyst in a trickle-bed reactor.

Khim. Prom-st. (Moscow) (11), 653-5 (1982) (Chem. Abstr. 98: 91383q) describes the preparation of morpholine by gas-phase cycloamination of diethylene glycol by means of ammonia in the presence of H₂ and a Cu, Co or Ni—Cr₂O₃ catalyst.

Zh. Vses. Khim. Obshchest. 14(5), 589-90 (1969) (Chem. Abstr. 72: 66879m) describes the formation of morpholine in a yield of 70% by gas-phase reaction of diethylene glycol with NH₃ over a nickel catalyst in the presence of H₂.

Ind. Eng. Chem. Prod. Res. Dev. 1981, 20, pages 399-407, (C. M. Barnes et al.) describes the ammonolysis of monoethanolamine (MEOA) to ethylenediamine (EDA) over nickel catalysts on a mixed SiO₂—Al₂O₃ support. Addition of water and the powdered catalyst are said to be advantageous in increasing the yield of EDA.

Disadvantages of these technologies involving suspension catalysis result, inter alia, from the need to separate the catalyst from the product. In addition, the selectivities, in particular for the formation of ADG, are in need of improvement.

A parallel German patent application filed on the same date (BASF AG) relates to a process for preparing ethylene amines by reaction of ethylenediamine (EDA) in the presence of specific shaped heterogeneous catalyst bodies.

A parallel German patent application filed on the same date (BASF AG) relates to a process for preparing ethylene amines by reacting monoethanolamine (MEOA) with ammonia in the presence of specific shaped heterogeneous catalyst bodies.

It is an object of the present invention to remedy the disadvantages of the prior art and discover an improved economical process for preparing aminodiglycol (ADG) and morpholine.

The process should, in particular, give the acyclic amine ADG of the formula

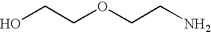

in high yields, space-time yields and selectivities.

For example, the proportion of ADG compared to morpholine in the product mix should be increased over that in the prior art, preferably at a high DEG conversion, in particular at a DEG conversion of greater than 85%.

[Space-time yields are reported in “amount of product/(catalyst volume·time)”(kg/(l_{cat}·h)) and/or “amount of product/(reactor volume·time)”(kg/(l_{reactor}·h)].

We have accordingly found a process for preparing aminodiglycol (ADG) and morpholine by reacting diethylene glycol (DEG) with ammonia in the presence of a heterogeneous transition metal catalyst, wherein the catalytically active composition of the catalyst before treatment with hydrogen comprises oxygen-comprising compounds of aluminum and/or zirconium, copper, nickel and cobalt and the shaped catalyst body has a diameter of <3 mm in the case of a spherical shape or extrudate form, a height of <3 mm in the case of a pellet shape and in the case of all other geometries in each case an equivalent diameter L=1/a′ of <0.70 mm, where a′ is the external surface area per unit volume (mmₛ²/mmₚ³), with:

$${a}^{′} = \frac{{A}_{p}}{{V}_{p}} ,$$

where Aₚ is the external surface area of the catalyst particle (mmₛ²) and Vₚ is the volume of the catalyst particle (mmₚ³).

The surface area and the volume of the catalyst particle (the shaped catalyst body) are derived from the geometric dimensions of the particle (shaped body) according to known mathematical formulae.

The volume can also be calculated by the following method, in which,
1. the internal porosity of the shaped body is determined (e.g. by measuring the water absorption in [ml/g of cat] at room temperature and a total pressure of 1 bar),
2. the displacement of the shaped body on immersion in a liquid is determined (e.g. by displacement of gas by means of a helium pycnometer) and
3. the sum of the two volumes is calculated.

The surface area can also be calculated theoretically by the following method, in which an envelope of the shaped body whose curve radii are not more than 5 μm (in order not to include the internal pore surface area by “intrusion” of the envelope into the pores) and which contacts the shaped body very intimately (no plane of section with the support) is defined. This would clearly correspond to a very thin film which is placed around the shaped body and a vacuum is then applied from the inside so that the film envelopes the shaped body very tightly.

The diethylene glycol (DEG) required as starting material can be prepared by known methods, for example by reacting ethylene oxide (EO) with H₂O or by reacting EO with monoethylene glycol.

The reaction according to the invention is generally carried out at an absolute pressure in the range 1-260 bar, preferably 100-250 bar, in particular 150-240 bar, very particularly preferably 175-225 bar, and generally at elevated temperature, e.g. in the temperature range 100-300° C., in particular 130-240° C., preferably 175-225° C.

DEG and ammonia are preferably used in a molar ratio in the range NH₃:DEG=1-15, particularly preferably in the range NH₃:DEG=4-13, very particularly preferably in the range NH₃:DEG=5-12.

The ratio of morpholine:ADG in the process of the invention is determined, in particular, by the DEG conversion and the molar ratio of NH₃:DEG.

In general, the catalysts used in the process of the invention are preferably used in the form of catalysts which either consist entirely of catalytically active composition and, if appropriate, a shaping aid (e.g. graphite or stearic acid) or are composed of the catalytically active components on a largely inactive support material.

The catalytically active composition can be introduced into the reaction vessel as powder or crushed material after milling or preferably be introduced into the reactor as shaped catalyst bodies, for example as pellets, spheres, rings, extrudates (e.g. rods, tubes) after milling, mixing with shaping aids, shaping and heat treatment.

The concentrations (in % by weight) indicated for the components of the catalyst are in each case, unless indicated otherwise, based on the catalytically active composition of the catalyst produced before treatment with hydrogen.

The catalytically active composition of the catalyst is defined as the sum of the masses of the catalytically active constituents and preferably comprises, before treatment with hydrogen, essentially the catalytically active constituents oxygen-comprising compounds of aluminum and/or zirconium, copper, nickel and cobalt.

The sum of the abovementioned catalytically active constituents, calculated as Al₂O₃, ZrO₂, CuO, NiO and CoO, in the catalytically active composition before treatment with hydrogen is, for example, from 70 to 100% by weight, preferably from 80 to 100% by weight, particularly preferably from 90 to 100% by weight, in particular from 95 to 100% by weight, very particularly preferably from >99 to 100% by weight.

Preferred heterogeneous catalysts in the process of the invention comprise, in their catalytically active composition before treatment with hydrogen,

from 20 to 85% by weight, preferably from 20 to 65% by weight, particularly preferably from 22 to 40% by weight, of Al₂O₃ and/or ZrO₂,
from 1 to 30% by weight, particularly preferably from 2 to 25% by weight, of oxygen-comprising compounds of copper, calculated as CuO,
from 14 to 70% by weight, preferably from 15 to 50% by weight, particularly preferably from 21 to 45% by weight, of oxygen-comprising compounds of nickel, calculated as NiO, with the molar ratio of nickel to copper preferably being greater than 1, in particular greater than 1.2, very particularly preferably from 1.8 to 8.5, and
from 15 to 50% by weight, particularly preferably from 21 to 45% by weight, of oxygen-comprising compounds of cobalt, calculated as CoO.

The oxygen-comprising compounds of copper, nickel and cobalt, in each case calculated as CuO, NiO and CoO, of the preferred catalysts are generally comprised in the catalytically active composition (before treatment with hydrogen) in total amounts of from 15 to 80% by weight, preferably from 35 to 80% by weight, particularly preferably from 60 to 78% by weight, with the molar ratio of nickel to copper particularly preferably being greater than 1.

Further heterogeneous catalysts which can be used in the process of the invention are

catalysts which are disclosed in DE-A-19 53 263 (BASF AG) and comprise cobalt, nickel and copper and aluminum oxide and have a metal content of from 5 to 80% by weight, in particular from 10 to 30% by weight, based on the total catalyst, with the catalyst comprising, calculated on the basis of the metal content, from 70 to 95% by weight of a mixture of cobalt and nickel and from 5 to 30% by weight of copper and the weight ratio of cobalt to nickel being from 4:1 to 1:4, in particular from 2:1 to 1:2, for example the catalyst used in the examples there which has the composition 10% by weight of CoO, 10% by weight of NiO and 4% by weight of CuO on Al₂O₃,
catalysts which are disclosed in EP-A-382 049 (BASF AG) or can be produced in an analogous manner and whose catalytically active composition before treatment with hydrogen comprises
from 20 to 85% by weight, preferably from 70 to 80% by weight, of ZrO₂ and/or Al₂O₃, from 1 to 30% by weight, preferably from 1 to 10% by weight, of CuO,
and in each case from 1 to 40% by weight, preferably from 5 to 20% by weight, of CoO and NiO,
for example the catalysts described in loc. cit. on page 6 which have the composition 76% by weight of Zr, calculated as ZrO₂, 4% by weight of Cu, calculated as CuO, 10% by weight of Co, calculated as CoO, and 10% by weight of Ni, calculated as NiO,
catalysts which are disclosed in EP-A-963 975 and EP-A-1 106 600 (both BASF AG) and whose catalytically active composition before treatment with hydrogen comprises from 22 to 40% by weight of ZrO₂,
from 1 to 30% by weight of oxygen-comprising compounds of copper, calculated as CuO,
from 15 to 50% by weight of oxygen-comprising compounds of nickel, calculated as NiO, with the molar ratio of Ni:Cu being greater than 1,
from 15 to 50% by weight of oxygen-comprising compounds of cobalt, calculated as CoO,
from 0 to 10% by weight of oxygen-comprising compounds of aluminum and/or manganese, calculated as Al₂O₃ or MnO₂,
and no oxygen-comprising compounds of molybdenum,
for example the catalyst A disclosed in loc. cit., page 17, which has the composition 33% by weight of Zr, calculated as ZrO₂, 28% by weight of Ni, calculated as NiO, 11% by weight of Cu, calculated as CuO, and 28% by weight of Co, calculated as CoO.

Catalysts which are particularly preferred in the process of the invention comprise no chromium (Cr).

The catalysts produced can be stored as such. Before use as catalysts in the process of the invention, they are prereduced (=activation of the catalyst) by treatment with hydrogen. However, they can also be used without prereduction, in which case they are then reduced (=activated) by the hydrogen present in the reactor under the conditions of the process of the invention.

To activate the catalyst, it is exposed to a hydrogen-comprising atmosphere or a hydrogen atmosphere at a temperature of preferably from 100 to 500° C., particularly preferably from 150 to 400° C., very particularly preferably from 180 to 300° C., for a period of at least 25 minutes, particularly preferably at least 60 minutes. The time for which the catalyst is activated can be up to 1 hour, particularly preferably up to 12 hours, in particular up to 24 hours.

During this activation, at least part of the oxygen-comprising metal compounds present in the catalysts is reduced to the corresponding metals, so that these are present together with the various oxygen compounds in the active form of the catalyst.

The catalyst used preferably has a bulk density in the range from 0.6 to 1.2 kg/l.

According to the invention, it has been noted that particularly high ADG selectivities are obtained when the catalyst is used in the form of small shaped bodies. For the purposes of the present invention, small shaped bodies are bodies whose diameter in the case of a spherical shape is in each case less than 3 mm, in particular less than 2.5 mm, e.g. in the range from 1 to 2 mm.

Correspondingly, small shaped bodies are also ones whose diameter in the case of extrudate form (extrudate length>>extrudate diameter) or whose height in the case of a pellet shape (pellet diameter>>pellet height) is in each case less than 3 mm, in particular less than 2.5 mm, e.g. in the range from 1 to 2 mm.

In the case of all other geometries, the shaped catalyst body used in the process of the invention in each case has an equivalent diameter L=1/a′ of <0.70 mm, in particular <0.65 mm, e.g. in the range from 0.2 to 0.6 mm, where a′ is the external surface area per unit volume (mmₛ²/mmₚ³), with:

$${a}^{′} = \frac{{A}_{p}}{{V}_{p}} ,$$

where Aₚ is the external surface area of the catalyst particle (mmₛ²) and Vₚ is the volume of the catalyst particle (mmₚ³).
(L=specific dimension of a shaped catalyst body).

In the process of the invention, the diffusion paths of the reactants and also of the products are shorter as a result of the small specific dimension of the catalyst particles. The mean residence time of the molecules in the pores and the probability of an undesirable subsequent reaction are consequently reduced. As a result of the defined residence time, an increased selectivity can be achieved, especially in the direction of the desired ADG.

The catalyst is preferably present as a fixed bed in a reactor. The reactor is preferably a tube reactor or a shell-and-tube reactor. The reaction of DEG is preferably carried out in a single pass through the reactor.

The bed of the catalyst is preferably surrounded with an inert material both at the entrance and at exit of the reactor. For example, Pairings of balls made from in inert material (for example, ceramics, steatite, aluminium) may be employed as inert material.

The reactor may be operated in both the sump and the trickling operation mode. In the preferred trickling operation mode, a liquid distributor is preferably employed for the reactor feed at the entrance of the reactor.

To maintain the catalyst activity, preference is given to feeding 0.01-1.00% by weight, particularly preferably 0.20-0.60% by weight, of hydrogen (based on the reactor feed DEG+NH₃) into the reactor.

In the preferred continuous operation, selectivities (S) to ADG and morpholine of preferably >60%, in particular 70-85%, are achieved at a conversion of 85-95% at an WHSV (weight hourly space velocity) of 0.25-2.0 kg/kg*h (kg of DEG per kg of cat. per hour), particularly preferably from 0.5 to 1.5 kg/kg*h. The molar selectivities to ADG+morpholine are very particularly preferably 90-92%.

At a DEG conversion of >90%, ADG and morpholine are typically formed in a weight ratio of ADG:morpholine of greater than 0.20, particularly preferably greater than 0.24, very particularly preferably greater than 0.27, e.g. in the range from 0.28 to 0.36.

As further products, small amounts of morpholine derivatives and higher amines, in particular higher linear polyalkylamines, are formed in the process of the invention.

The work-up of the product streams obtained in the process of the invention, which, in particular, comprise the particularly desired ADG but also morpholine, morpholine derivatives, higher polyalkylamines and unreacted DEG, can be carried out by distillation processes known to those skilled in the art.

The distillation columns required for isolating the individual products, especially the particularly desired ADG and also morpholine, in pure form by distillation can be designed (e.g. number of theoretical plates, reflux ratio, etc.) by those skilled in the art using methods with which they would be familiar.

The fractionation of the reaction product mixture resulting from the reaction is, in particular, carried out by multistage distillation.

For example, the fractionation of the reaction product mixture resulting from the reaction is carried out by multistage distillation in two separation sequences, with ammonia and any hydrogen present being separated off first in the first separation sequence and fractionation into unreacted DEG and ADG, morpholine, morpholine derivatives and higher polyalkylamines being carried out in the second separation sequence.

The ammonia obtained from the reaction product mixture resulting from the reaction from the fractionation and/or DEG obtained are/is preferably recirculated to the reaction.

### EXAMPLES

### A Production of Catalyst

### A1 Preparation of Precursor

To carry out the precipitation, an aqueous solution of nickel nitrate, copper nitrate, cobalt nitrate and zirconium acetate was introduced at a constant flow rate together with a 20% strength aqueous sodium carbonate solution into a stirred vessel at a temperature of 70° C. in such a way that the pH was maintained in the range 5.5-6.0. After completion of the addition of the metal salt solution and the sodium carbonate solution, the mixture was stirred for another one hour at 70° C. and the pH was subsequently increased to 7.4 by addition of a little sodium carbonate solution.

The suspension obtained was filtered and the filter cake was washed with deionized water. The filter cake was then dried at a temperature of 200° C. in a drying oven or a spray drier. The hydroxide/carbonate mixture obtained in this way was then heated at a temperature of 400° C. for a period of 2 hours.

The catalyst powder obtained in this way had the composition:

28.1% by weight of Ni, calculated as NiO
27.7% by weight of Co, calculated as CoO
13.1% by weight of Cu, calculated as CuO
31.2% by weight of Zr, calculated as ZrO₂

### A2 Catalyst A (Comparative Catalyst)

The catalyst powder from A1 was mixed with 2% by weight of graphite and shaped to produce 5×3 mm pellets. After tableting, the pellets were after-calcined at 350° C. for 2 hours in a muffle furnace. Before installation in the test reactor, it was reduced and subsequently passivated: to reduce the catalyst, it was heated in a stream of hydrogen/nitrogen at temperatures of from 100 to 200° C. This temperature was maintained until no more water was formed. The catalyst was subsequently heated to a final temperature of 280° C. and this temperature was maintained for 90-120 hours. The catalyst was cooled to room temperature under a stream of nitrogen and then passivated by means of a diluted stream of oxygen. During the passivation, care was taken to ensure that the temperature did not exceed 50° C. at any point in the reactor.

### A3 Catalyst B (According to the Invention)

The catalyst powder from A1 was mixed with 2% by weight of graphite and shaped to produce 1.5×2 mm pellets. After-calcination, reduction and passivation were carried out as described in A2.

### B Hydrogenative Amination Using Catalysts as Described in A

### Example 1

### Small Shaped Body (Catalyst B)

### According to the Invention DEG (700 g/h), NH₃ (730 g/h) and H₂ (90 standard l/h) (standard i=standard liters=volume at STP) were fed continuously in the upflow mode into a stainless steel tube (length: 2 m, diameter: 3 cm). The reactor was filled with the amination catalyst (500 ml as 1.5×2 mm shaped bodies) and the reaction was carried out at 200 bar. The space velocity over the catalyst was 1.4 kg/l*h.

At 192° C., the following were obtained:

DEG: 29.6% by weight
ADG: 31.4% by weight
Morpholine; 32.1% by weight

At 195° C., the following were obtained:

DEG: 19.3% by weight
ADG: 28.7% by weight
Morpholine: 43.7% by weight

At 198° C., the following were obtained:

DEG: 9.1% by weight
ADG: 20.6% by weight
Morpholine: 60.2% by weight

### Comparative Example 1

### Classical Shaped Body

### Catalyst A

DEG (700 g/h), NH₃ (730 g/h) and H₂ (90 standard l/h) were fed continuously in the upflow mode into a stainless steel tube (length: 2 m, diameter: 3 cm). The reactor was filled with the amination catalyst (500 ml as 5×3 mm shaped bodies) and the reaction was carried out at 195° C. and 200 bar. The space velocity over the catalyst was 1.4 kg/l*h.

The following product mix was obtained:

DEG: 22.8% by weight
ADG: 22.5% by weight
Morpholine: 46.9% by weight

1.-17. (canceled)

18. A process comprising; providing a starting material comprising diethylene glycol; and reacting the starting material with ammonia in the presence of a heterogeneous transition metal catalyst to form a reaction product comprising aminodiglycol and morpholine; wherein the catalyst comprises a catalytically active composition, which prior to treatment with hydrogen, comprises a mixture of oxygen-containing compounds of copper, nickel, cobalt and at least one of aluminum and zirconium; and wherein the catalyst is present as one or more shaped catalyst particles selected from spheres, extrudates, pellets and other geometries, wherein the sphere or extrudate has a diameter of <3 mm, the pellet has a height of <3 mm, and the other geometries have an equivalent diameter L=1/a′ of <0.70 mm, where a′ is the external surface area per unit volume (mmₛ²/mmₚ³), as defined by
$${a}^{′} = \frac{{A}_{p}}{{V}_{p}}$$
where Aₚ is the external surface area of the catalyst particle (mmₛ²) and Vₚ is the volume of the catalyst particle (mmₚ³).

19. The process according to claim 18, wherein the aminodiglycol and morpholine are formed in a weight ratio of aminodiglycol:morpholine of greater than 0.20.

20. The process according to claim 18, wherein the sphere or extrudate has a diameter of <2.5 mm, the pellet has a height of <2.5 mm, and the other geometries have an equivalent diameter L=1/a′ of <0.65 mm.

21. The process according to claim 18, wherein reacting the starting material is carried out in the further presence of hydrogen.

22. The process according to claim 18, wherein reacting the starting material is carried out at a temperature of 100 to 300° C.

23. The process according to claim 18, wherein reacting the starting material is carried out at an absolute pressure of 10 to 200 bar.

24. The process according to claim 18, wherein reacting the starting material is carried out in the gas phase, in the liquid phase, or a supercritical phase.

25. The process according to claim 18, wherein the catalytically active composition, prior to treatment with hydrogen, comprises 20 to 65% by weight of zirconium dioxide, 1 to 30% by weight of one or more oxygen-containing compounds of copper, calculated as CuO, 15 to 50% by weight of one or more oxygen-containing compounds of nickel, calculated as NiO, and 15 to 50% by weight of one or more oxygen-containing compounds of cobalt, calculated as CoO.

26. The process according to claim 18, wherein the catalyst has a bulk density of 0.6 to 1.2 kg/l.

27. The process according to claim 18, wherein reacting the starting material is carried out in a reactor, and the catalyst is present in the reactor as a fixed bed.

28. The process according to claim 27, wherein the reactor is selected from the group consisting of tube reactors and shell-and-tube reactors.

29. The process according to claim 27, wherein reacting the starting material is carried out in a single pass through the reactor.

30. The process according to claim 27, wherein the reactor is operated in the sump operation mode or in the trickling operation mode.

31. The process according to claim 18, wherein the diethylene glycol and the ammonia are reacted in a molar ratio of ammonia:diethylene glycol of 1 to 15.

32. The process according to claim 18, further comprising fractionating the reaction product in a multistage distillation.

33. The process according to claim 32, wherein the multistage distillation comprises a first separation sequence and a second separation sequence, wherein ammonia and hydrogen present in the reaction product are separated from a remainder of the reaction product in the first separation sequence, and wherein unreacted diethylene glycol, aminodiglycol and morpholine, and optionally any morpholine derivatives and other higher polyalkylamines present in the remainder of the reaction product, are fractionated in the second separation sequence.

34. The process according to claim 33, wherein one or both of the ammonia obtained from the first separation sequence and the unreacted diethylene glycol obtained from the second separation sequence is recirculated to the reaction.

35. The process according to claim 18, wherein the one or more shaped catalyst particles comprises a pellet.

36. A process comprising: providing a starting material comprising diethylene glycol; and reacting the starting material with ammonia in the presence of a heterogeneous transition metal catalyst to form a reaction product comprising aminodiglycol and morpholine; wherein the catalyst comprises a catalytically active composition, which prior to treatment with hydrogen, comprises a mixture of oxygen-containing compounds of copper, nickel, cobalt and at least one of aluminum and zirconium; and wherein the catalyst is present as one or more shaped catalyst particles selected from spheres, extrudates, pellets and other geometries, wherein the sphere or extrudate has a diameter of <2.5 mm, the pellet has a height of <2.5 mm, and the other geometries have an equivalent diameter L=1/a′ of <0.70 mm, where a′ is the external surface area per unit volume (mmₛ²/mmₚ³), as defined by
$${a}^{′} = \frac{{A}_{p}}{{V}_{p}}$$
where Aₚ is the external surface area of the catalyst particle (mmₛ²) and Vₚ is the volume of the catalyst particle (mmₚ³).

37. The process according to claim 36, wherein the other geometries have an equivalent diameter L=1/a′ of <0.65 mm.
